(19) United States
(12) Patent Application Publication
(10) Pub. No.: US 2014/0045214 A1
(43) Pub. Date: Feb. 13, 2014

(54) MUTANT MICROORGANISM BELONGING TO THE GENUS TRICHODERMA AND METHOD FOR PRODUCING PROTEIN USING THE SAME

(71) Applicants: Ai Nishida, Toyota-shi (JP); Katsunori Kohda, Nisshin-shi (JP); Wataru Ogasawara, Nagaoka-shi (JP)

(72) Inventors: Ai Nishida, Toyota-shi (JP); Katsunori Kohda, Nisshin-shi (JP); Wataru Ogasawara, Nagaoka-shi (JP)

(73) Assignee: TOYOTA JIDOSHA KABUSHIKI KAISHA, Toyota-shi, Aichi (JP)

(21) Appl. No.: 14/113,627

(22) PCT Filed: Apr. 20, 2012
(86) PCT No.: PCT/JP2012/002755

§ 371 Date: Oct. 24, 2013

(30) Foreign Application Priority Data
Apr. 27, 2011 (JP) 2011-099956
Nov. 17, 2011 (JP) 2011-252004

(51) Int. Cl. C07K 14/415; C12N 15/80

(52) U.S. Cl. CPC C07K 14/415; C12N 15/80
USPC 435/69.1; 435/254.6

(57) ABSTRACT

An object of the present invention is to provide a mutant microorganism belonging to the genus Trichoderma capable of secretory production of a protein of interest using a microorganism belonging to the genus Trichoderma as a host. The mutant microorganism belonging to the genus Trichoderma of the present invention is obtained by transforming a microorganism belonging to the genus Trichoderma with a polynucleotide encoding a maize-derived expansin signal peptide and a protein of interest.

### TECHNICAL FIELD

The present invention relates to a mutant microorganism belonging to the genus Trichoderma transformed with a polynucleotide encoding a predetermined protein and a method for producing a protein using the same.

### BACKGROUND ART

Microorganisms belonging to the genus Trichoderma including Trichoderma reesei carry out secretory production of biomass degradative enzymes such as cellulase. Thus, the microorganisms are known to be useful for biofuel production using plant biomass as starting material. In addition, it has been actively attempted to improve the cellulase production capacity of a microorganism belonging to the genus Trichoderma or to develop a highly functional mutant microorganism belonging to the genus Trichoderma capable of inducing expression of a variety of genes.

Specifically, Patent Document 1 discloses a method for carrying out secretory production of swollenin by linking the swollenin gene downstream of the CBH1 signal peptide and the CBH1 activation domain and causing expression of the swollenin gene in a filamentous fungus belonging to the genus Trichoderma or Aspergillus. In addition, the CBH1 signal peptide corresponds to a signal peptide of cellobiohydrolase 1. According to the method disclosed in Patent Document 1, it is understandable that the CBH1 signal peptide is useful for secretory production of a protein of interest using a microorganism belonging to the genus Trichoderma as a host.

However, the method described in Patent Document 1 is not a method whereby secretory expression of an arbitrary gene can be carried out. Thus, for example, secretory production of a plant-derived protein cannot be achieved thereby, which is problematic. There are not many experimental examples of secretory production of a variety of proteins such as plant-derived proteins. For example, one of such known examples is described in Non-Patent Document 1. Non-Patent Document 1 discloses that expression of barley-derived endopeptidase was induced using Trichoderma reesei as a host.

Development of a technique whereby secretory production of a variety of proteins can be carried out using microorganisms belonging to the genus Trichoderma as hosts has been awaited.

Citation List

Patent Literature

PTL 1: WO2007/115723

Non Patent Literature

NPL 1: Applied and Environmental Microbiology, 1997, 63 (12), pp. 4938-4940

### SUMMARY OF INVENTION

Technical Problem

In view of the above circumstances, an object of the present invention is to provide a mutant microorganism belonging to the genus Trichoderma capable of secretory production of a protein of interest using a microorganism belonging to the genus Trichoderma as a host. Another object of the present invention is to provide a method for producing the protein of interest using the mutant microorganism belonging to the genus Trichoderma.

Solution to Problem

The present inventors conducted intensive studies to achieve the above objects. Accordingly, the present inventors found that secretory production of a gene product can be achieved in a microorganism belonging to the genus Trichoderma using a signal peptide contained in the maize (Zea mays)-derived expansin gene. This has led to the completion of the present invention.

The present invention encompasses the following (1) to (5).

(1) A mutant microorganism belonging to the genus Trichoderma, which is obtained by transforming a microorganism belonging to the genus Trichoderma with a polynucleotide encoding a maize-derived expansin signal peptide and a protein of interest.

(2) The mutant microorganism belonging to the genus Trichoderma according to (1), wherein the microorganism belonging to the genus Trichoderma is Trichoderma reesei.

(3) The mutant microorganism belonging to the genus Trichoderma according to (1), wherein the signal peptide comprises the amino acid sequence shown in SEQ ID NO: 1.

(4) The mutant microorganism belonging to the genus Trichoderma according to (1), wherein the protein of interest is a maize-derived expansin.

(5) The mutant microorganism belonging to the genus Trichoderma according to (1), wherein the protein of interest is cellobiohydrolase, which cleaves a cellulose chain from the nonreducing end in cellobiose-unit-sized pieces.

(6) The mutant microorganism belonging to the genus Trichoderma according to (1), wherein the protein of interest is an antibody.

(7) A method for producing a protein of interest comprising culturing the mutant microorganism belonging to the genus Trichoderma according to any one of (1) to (6) above in a medium so as to cause secretory production of the protein of interest.

Advantageous Effects of Invention

According to the present invention, the use of a maize-derived expansin signal peptide allows a microorganism belonging to the genus Trichoderma used as a host to carry out secretory production of a variety of proteins. Specifically, the mutant microorganism belonging to the genus Trichoderma of the present invention comprises a maize-derived expansin signal peptide that functions to cause secretory production of a protein of interest. Therefore, a method for producing a protein with excellent production efficiency can be provided using the mutant microorganism belonging to the genus Trichoderma of the present invention.

### BRIEF DESCRIPTION OF DRAWINGS

FIG. 1 schematically shows the configuration of the expression vector produced in Example 1.

FIG. 2 shows an SDS-PAGE image and a Western blotting image each indicating results obtained for the culture supernatant of a transformant produced in Example 1.

FIG. 3 shows a Western blotting image indicating results for the cell disruption solution and the culture supernatant of the transformant produced in Comparative Example 1.

FIG. 4 shows a Western blotting image indicating results for the cell disruption solution and the culture supernatant of the transformant produced in Comparative Example 2.

FIG. 5 is a characteristic chart showing results of evaluation of saccharification ability for the transformant produced in Example 1.

FIG. 6 shows an SDS-PAGE image and a Western blotting image each indicating results obtained for the culture supernatant of a transformant (the Trichoderma reesei TU6 strain) produced in Example 1.

FIG. 7 shows an SDS-PAGE image and a Western blotting image each indicating results obtained for the culture supernatant of a transformant (the Trichoderma reesei PC3-7 strain) produced in Example 2.

FIG. 8 shows an SDS-PAGE image and a Western blotting image each indicating results obtained for the culture supernatant of a transformant (the Trichoderma reesei TU6 strain) produced in Example 3.

FIG. 9 shows an ELISA image indicating results obtained for the culture supernatant of a transformant (the Trichoderma reesei TU6 strain) produced in Example 3.

### DESCRIPTION OF EMBODIMENTS

The mutant microorganism belonging to the genus Trichoderma and the method for producing a protein using the same according to the present invention are described in detail below.

The mutant microorganism belonging to the genus Trichoderma according to the present invention is obtained by introducing a polynucleotide encoding a maize-derived expansin signal peptide and a protein of interest into a microorganism belonging to the genus Trichoderma used as a host.

<Microorganisms Belonging to the Genus Trichoderma>

Microorganisms belonging to the genus Trichoderma used herein include all filamentous microorganisms classified as belonging to the genus Trichoderma. A microorganism belonging to the genus Trichoderma used as a host in the present invention may be the wild-type strain of a conventionally known microorganism belonging to the genus Trichoderma or it may be a mutant strain derived from the wild-type strain. Such mutant strain may be a strain mutated via gene introduction, gene disruption, or the like or it may be a strain mutated via UV irradiation or mutagenic treatment using a mutagen such as a nitroso compound. Examples of microorganisms belonging to the genus Trichoderma include Trichoderma aggressivum, Trichoderma asperellum, Trichoderma atroviride, Trichoderma aureoviride, Trichoderma austrokoningii, Trichoderma brevicompactum, Trichoderma candidum, Trichoderma caribbaeum var. aequatoriale, Trichoderma caribbaeum var. caribbaeum, Trichoderma catoptron, Trichoderma cremeum, Trichoderma ceramicum, Trichoderma cerinum, Trichoderma chlorosporum, Trichoderma chromospermum, Trichoderma cinnamomeum, Trichoderma citrinoviride, Trichoderma crassum, Trichoderma cremeum, Trichoderma dingleyeae, Trichoderma dorotheae, Trichoderma effusum, Trichoderma erinaceum, Trichoderma estonicum, Trichoderma fertile, Trichoderma gelatinosus, Trichoderma ghanense, Trichoderma hamatum, Trichoderma harzianum, Trichoderma helicum, Trichoderma intricatum, Trichoderma konilangbra, Trichoderma koningii, Trichoderma koningiopsis, Trichoderma longibrachiatum, Trichoderma longipile, Trichoderma minutisporum, Trichoderma oblongisporum, Trichoderma ovalisporum, Trichoderma petersenii, Trichoderma phyllostahydis, Trichoderma piluliferum, Trichoderma pleuroticola, Trichoderma pleurotum, Trichoderma polysporum, Trichoderma pseudokoningii, Trichoderma pubescens, Trichoderma reesei, Trichoderma rogersonii, Trichoderma rossicum, Trichoderma saturnisporum, Trichoderma sinensis, Trichoderma sinuosum, Trichoderma sp. MA 3642, Trichoderma sp. PPRI 3559, Trichoderma spirale, Trichoderma stramineum, Trichoderma strigosum, Trichoderma stromaticum, Trichoderma surrotundum, Trichoderma taiwanense, Trichoderma thailandicum, Trichoderma thelephoricolum, Trichoderma theobromicola, Trichoderma tomentosum, Trichoderma velutinum, Trichoderma virens, Trichoderma viride, and Trichoderma viridescens. Of these, it is preferable to use, as a host, a microorganism belonging to the genus Trichoderma, which is highly capable of secretory production of cellulase. An example of such microorganism belonging to the genus Trichoderma that is highly capable of secretory production of cellulase is Trichoderma reesei. Specifically, it is preferable to use Trichoderma reesei as the host according to the present invention.

<Signal Peptide>

According to the present invention, a maize-derived expansin signal peptide is used as a signal peptide that functions to cause extracellular secretion of a protein of interest. The maize-derived expansin gene is disclosed in US Patent Publication No. 2002/0103355. The amino acid sequence of the maize-derived expansin signal peptide is shown in SEQ ID NO: 1. In addition, the nucleotide sequence of a polynucleotide encoding the amino acid sequence shown in SEQ ID NO: 1 can be adequately designed. In particular, in order to improve translation efficiency of a microorganism belonging to the genus Trichoderma, it is preferable to design a nucleotide sequence of a polynucleotide encoding the amino acid sequence shown in SEQ ID NO: 1 using a codon that is frequently used for a microorganism belonging to the genus Trichoderma.

<Protein of Interest>

According to the present invention, extracellular secretory production of a protein of interest can be carried out by fusing the protein of interest to the above signal peptide. Such protein of interest used herein is not limited and thus it may be a protein from an arbitrary organism species having an arbitrary molecular weight, isoelectric point, or amino acid sequence. If a protein of interest is a protein from a non-maize organism, a polynucleotide encoding a fusion protein comprising the above signal peptide and the protein of interest is introduced into a microorganism belonging to the genus Trichoderma.

In addition, the protein of interest may be a maize-derived expansin. When the protein of interest is a maize-derived expansin, the maize-derived expansin gene comprising a region encoding the signal peptide is introduced into a microorganism belonging to the genus Trichoderma.

In particular, an example of the protein of interest is an enzyme involved in saccharification of plant biomass. Microorganisms belonging to the genus Trichoderma have been used for saccharification of plant biomass because they can carry out extracellular secretory production of cellulase. Therefore, saccharification efficiency of plant biomass can be improved by introducing, as a protein of interest, such enzyme involved in saccharification into a microorganism belonging to the genus Trichoderma. Here, the expansin is an enzyme having an activity of cleaving a hydrogen bond between cellulose and a matrix in a plant. Further, an enzyme involved in saccharification of biomass is not limited to the expansin. Examples of such enzyme include cellobiohydrolase, endoglucanase, and beta-glucosidase. That is, examples of a protein of interest include cellobiohydrolase, endoglucanase, and beta-glucosidase. In addition, there exist the following types of cellobiohydrolase: cellobiohydrolase I which cleaves cellulose from the reducing end in cellobiose-unit-sized pieces; and cellobiohydrolase II which cleaves cellulose from the nonreducing end in cellobiose-unit-sized pieces. The protein of interest may be either cellobiohydrolase I or II.

Meanwhile, the origin of the protein of interest may be a protein from an arbitrary organism species such as a virus, an archaebacterium, a bacterium, a fungus, an actinomycete, an algae, a protozoa, an insect, a plant, or an animal. An example of the protein of interest is an antibody. In this case, a system for producing an antibody can be constructed using the aforementioned microorganism belonging to the genus Trichoderma.

<Vector>

The polynucleotide encoding the signal peptide and the protein of interest described above can be incorporated into a generally available expression vector so as to be introduced into a microorganism belonging to the genus Trichoderma. A typical example of such vector has a selectable marker gene, a cloning site, and control regions (corresponding to a promoter and a terminator). Such vector is well known in the art and commercially available.

A promoter that can be contained in a vector may be either a constitutive expression promoter or an inducible promoter as long as it can function in a microorganism belonging to the genus Trichoderma. A situation in which a promoter can function in the above manner refers to a situation in which transcription of a downstream gene is possible in a host microorganism (a microorganism belonging to the genus Trichoderma in this case). For example, a promoter of a gene encoding glucoamylase, alpha-amylase, or alpha-glucosidase from a microorganism belonging to the genus Aspergillus such as Aspergillus niger can be used as a promoter. Also, the following can be used: a promoter of the gpdA gene, the oliC gene, or the trpC gene of A. nidulans; a promoter of the cbh1 or trp1 gene of Neurospora crassa; a promoter of the aspartic proteinase-encoding gene of A. niger or Rhizomucor miehei; and a promoter of the cbh1 gene, the cbh2 gene, the eg11 gene, the eg12 gene, or a gene encoding a different cellulase from Trichoderma reesei. In particular, when a microorganism belonging to the genus Trichoderma to be used as a host is Trichoderma reesei, it is preferable to use, as the above promoter, a promoter of the cbh1 gene, the cbh2 gene, the eg11 gene, the eg12 gene, or a gene encoding a different cellulase from Trichoderma reesei. In addition, it is more preferable to use a promoter of the cbh1 gene.

<Production of Protein of Interest>

According to the present invention, an expression vector into which the polynucleotide encoding the signal peptide and the protein of interest described above have been incorporated is introduced into a microorganism belonging to the genus Trichoderma by a conventional method so as to cause extracellular secretory production of the protein of interest. Thus, the protein of interest can be collected outside the cells of a microorganism belonging to the genus Trichoderma. The protein of interest in a cell lysate or a medium supernatant can be detected by western blotting using sodium dodecyl sulfate polyacrylamide gel electrophoresis (SDS-PAGE) known in the art. A protein of interest produced in a cell culture is secreted into a medium. Then, the protein is purified or isolated by, for example, removing unnecessary components from the cell medium. For example, the following techniques can be used alone or in combination for purification of the protein of interest: affinity chromatography, ion-exchange chromatography, hydrophobic interaction chromatography, ethanol precipitation, reversed-phase HPLC, chromatography performed on silica or with a cation-exchange resin such as DEAE, chromatofocusing, SDS-PAGE, ammonium sulfate precipitation, and gel filtration.

### EXAMPLES

The present invention is hereafter described in greater detail with reference to the following examples, although the technical scope of the present invention is not limited thereto.

### Example 1

In this Example, a maize-derived expansin gene was introduced into Trichoderma reesei.

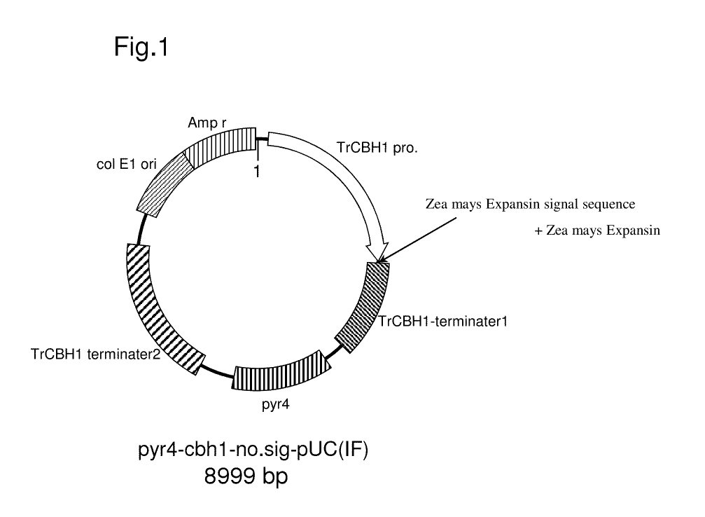

In this Example, a vector incorporating a maize-derived expansin gene was constructed (FIG. 1). The vector named as pyr4-cbh1-no.sig-pUC(IF) (shown in FIG. 1) was obtained by inserting the upstream region of the CBH1 gene including a CBH1 promoter at the KpnI and AatII sites of a pUC 19 vector, inserting the downstream region of the CBH1 gene including the CBH1 terminator at the KpnI and SphI sites of the pUC19 vector, and inserting the pyr4 sequence at the EcoRV and BamHI sites of the pUC19 vector.

In addition, the entire region of the maize-derived expansin gene including a region encoding the signal peptide was chemically synthesized in this Example. At this time, the nucleotide sequence of the maize-derived expansin gene was designed so as to use codons, which are frequently used for Trichoderma reesei used as a host. The nucleotide sequence of the region encoding the signal peptide region of the designed maize-derived expansin gene is shown in SEQ ID NO: 2. Further, the amino acid sequence of a region obtained by excluding the signal peptide region from the entire region of maize-derived expansin is shown in SEQ ID NO: 3.

The chemically synthesized maize-derived expansin gene was inserted at the KpnI and Spel sites of the above vector. Then, the vector was formed into a linear vector by restriction enzyme treatment using Sse8387I, followed by transformation of Trichoderma reesei. In this Example, the Trichoderma reesei PC3-7 strain was used as a host. Transformation of Trichoderma reesei was carried out by preparing a Trichoderma reesei protoplast and adding the above linear vector and PEG buffer thereto in such order according to a conventional method. Introduction of the maize-derived expansin gene into the obtained transformant was confirmed by colony PCR. Thereafter, the transformant was cultured in a cellulase production medium at 120 rpm and 28 degrees C. for 5 days. Accordingly, secretory production of the desired expansin was confirmed by SDS-PAGE and Western blotting.

Further, in this Example, it was attempted to induce secretory production of the maize-derived expansin gene using the CBH1 signal peptide (SEQ ID NO: 4) instead of the maize-derived expansin signal peptide for comparison. Specifically, Trichoderma reesei was transformed using a polynucleotide encoding a fusion protein consisting of the CBH1 signal peptide and the maize-derived expansin such that a transformant was produced for a comparative example (Comparative Example 1). Trichoderma reesei was transformed using a polynucleotide encoding a fusion protein comprising the CBH1 signal peptide and the maize-derived expansin bound downstream of the CBH1 activation domain such that a transformant was produced for another comparative example (Comparative Example 2).

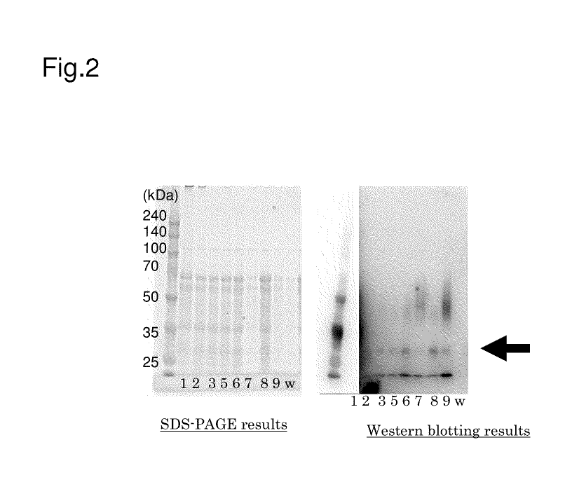

FIG. 2 shows SDS-PAGE analysis results and Western blotting analysis results for the culture supernatant of the transformant produced in Example 1. In addition, FIG. 2 shows results of Western blotting analysis using His6 tag antibodies. Further, “W” indicates the results for the culture supernatant of the wild-type Trichoderma reesei in FIG. 2.

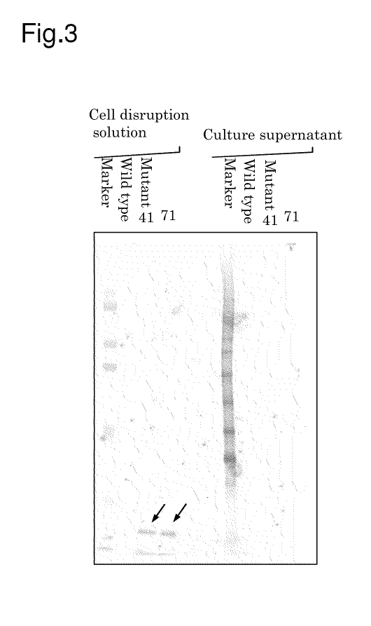

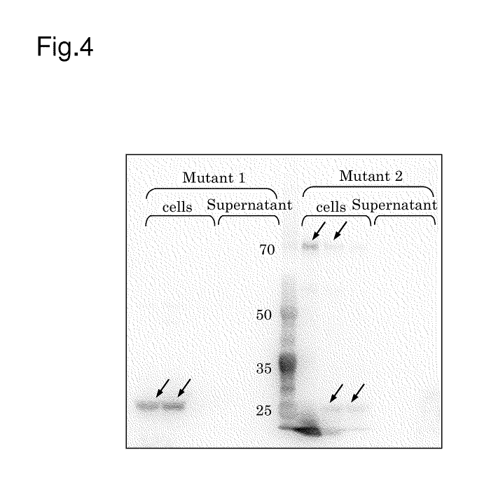

Further, FIG. 3 shows the results of evaluation by Western blotting for the cultured cells and the culture supernatant of the transformant produced in Comparative Example 1. Similarly, FIG. 4 shows the results of evaluation by Western blotting for the cultured cells and the culture supernatant of the transformant produced in Comparative Example 2. As shown in FIGS. 3 and 4, it was possible to confirm a band at the position corresponding to the protein of interest upon evaluation of the cell disruption solutions of the transformants produced in Comparative Examples 1 and 2. However, it was revealed that the protein of interest was not contained in the culture supernatants of the same. The results indicate that the transformants produced in Comparative Examples 1 and 2 cannot secrete a protein of interest while the protein can be intracellularly expressed therein. This suggests the CBH1 signal peptide does not function in the transformants.

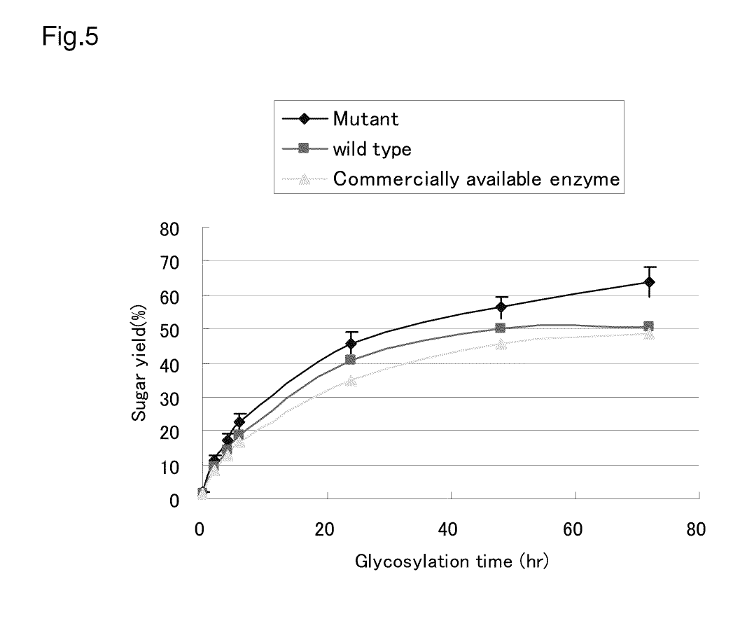

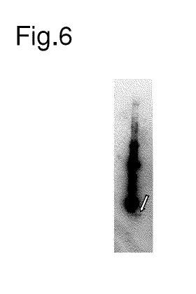

As a result of comparison between the transformant of Example 1 and the transformants of Comparative Examples 1 and 2, it was found that the protein of interest was able to be detected in the culture supernatant of the transformant of Example 1 (indicated by arrows in FIG. 2). The results revealed that the maize-derived expansin signal peptide can be effectively used for secretory production of a protein of interest in a microorganism belonging to the genus Trichoderma such as Trichoderma reesei. In addition, the transformant produced in Example 1 can carry out extracellular secretory production of an expansin. Thus, it is expected that the transformant produced in Example 1 is superior to the wild-type Trichoderma reesei in terms of the ability to degradate plant biomass. Therefore, the ability of the transformant produced in Example 1 was compared with that of the wild-type Trichoderma reesei or a commercially available enzyme used instead of the transformant. Specifically, first, the culture supernatants of the transformant, the wild-type Trichoderma reesei, and a commercially available enzyme were separately added to 1% Avicel such that the protein concentration became 6.7 mg/g per unit biomass. Novol88 (5 microliters) was added thereto for saccharification (50 mM sodium acetate buffer, 32 degrees C., 120 rpm). Sampling was performed at predetermined time points, followed by quantitative analysis of the glucose amount using a biosensor (Oji Scientific Instruments). FIG. 5 shows the results. As shown in FIG. 5, the transformant produced in Example 1 has the saccharification ability superior to that of the wild-type Trichoderma reesei. The results show that the transformant produced in Example 1 can induce secretory production of an expansin and has excellent saccharification ability. Therefore, it was revealed that the transformant produced in Example 1 is more preferable than the wild-type Trichoderma reesei for saccharification treatment. In addition, in this Example, it was attempted to induce secretory production of a maize-derived expansin using the Trichoderma reesei TU6 strain as a host in the manner described above. The experiment was performed in the manner described above except that the Trichoderma reesei TU6 strain was used as a host. FIG. 6 shows the results. As shown in FIG. 6, it was also revealed that the signal peptide contained in the maize-derived expansin can sufficiently function as a secretory signal in the Trichoderma reesei TU6 strain.

### Example 2

In this Example, secretory production of a protein of interest was examined using a signal peptide contained in a maize-derived expansin. Specifically, secretory production of white-rot-fungus (Phanerochaete chrysosporium)-derived CBH2 used as a protein of interest was examined in this Example.

First, a polynucleotide encoding a fusion protein obtained by fusing a signal peptide contained in a maize-derived expansin (SEQ ID NO: 1) and a region of CBH2 excluding a signal peptide was chemically synthesized. At this time, the nucleotide sequence of the synthesized polynucleotide was designed so as to use codons, which are frequently used for Trichoderma reesei used as a host. In addition, the nucleotide sequence shown in SEQ ID NO: 2 was used as the nucleotide sequence of a region encoding the signal peptide region of a maize-derived expansin as in the case of Example 1. Further, the nucleotide sequence of a region of the Phanerochaete chrysosporium-derived CBH2 gene excluding the signal peptide region designed so as to use codons, which are frequently used for Trichoderma reesei, is shown in SEQ ID NO: 5. Also, the amino acid sequence of a region of Phanerochaete chrysosporium-derived CBH2 excluding the signal peptide region is shown in SEQ ID NO: 6. Here, a motif consisting of 6 His residues on the N-terminal side of the amino acid sequence shown in SEQ ID NO: 6 is a so-called His6 tag.

In this Example, the vector used in Example 1, pyr4-cbh1-no.sig-pUC(IF), was also used. The chemically synthesized polynucleotide encoding a fusion protein was inserted at the KpnI and Spel sites of the above vector. Then, the vector was formed into a linear vector by restriction enzyme treatment using Sse8387I as in the case of Example 1, followed by transformation of the Trichoderma reesei PC3-7 strain. Further, in this Example, it was attempted to induce secretory production of the Phanerochaete chrysosporium-derived CBH2 gene using the Phanerochaete chrysosporium-derived CBH2 signal peptide (SEQ ID NO: 7) instead of the maize-derived expansin signal peptide for comparison. Specifically, the Phanerochaete chrysosporium-derived CBH2 gene was designed so as to encode Phanerochaete chrysosporium-derived CBH2 including a signal peptide and so as to use codons, which are frequently used for Trichoderma reesei. The nucleotide sequence encoding the signal peptide of Phanerochaete chrysosporium-derived CBH2 (SEQ ID NO: 7) designed to correspond to a codon, which is frequently used for Trichoderma reesei, is shown in SEQ ID NO: 8.

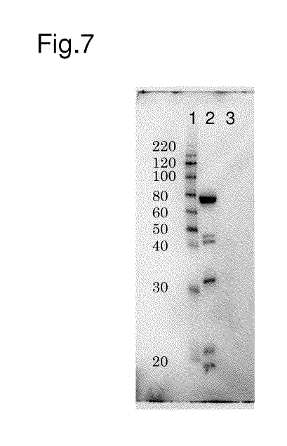

In addition, FIG. 7 shows Western blotting analysis and SDS-PAGE analysis results for the culture supernatant obtained by culturing transformed Trichoderma reesei as in the case of Example 1. In addition, Western blotting analysis shown in FIG. 7 was carried out using an His6-tag antibody. Further, in FIG. 7, lane 1 corresponds to a marker, lane 2 corresponds to the result for the culture supernatant of transformed Trichoderma reesei produced in this Example, and lane 3 corresponds to the result of the culture supernatant of transformed Trichoderma reesei produced for comparison.

As shown in FIG. 7, it was found that the culture supernatant of the transformant produced for comparison contained no Phanerochaete chrysosporium-derived CBH2. The results suggested that the signal peptide contained in Phanerochaete chrysosporium-derived CBH2 does not function as a secretory signal in Trichoderma reesei used as a host. On the other hand, the culture supernatant of transformed Trichoderma reesei produced in this Example was found to contain Phanerochaete chrysosporium-derived CBH2. The results revealed that the signal peptide contained in the maize-derived expansin can sufficiently function as a secretory signal in a microorganism belonging to the genus Trichoderma such as Trichoderma reesei used as a host.

### Example 3

In this Example, secretory production of a protein of interest was examined using a signal peptide contained in a maize-derived expansin. Specifically, in this Example, secretory production of anti-egg-white lysozyme camelid single-domain VHH antibodies (1ZV5) used as a protein of interest was examined.

Also for the anti-egg-white lysozyme camelid single-domain VHH antibodies (1ZV5), a polynucleotide encoding a fusion protein obtained by fusing a signal peptide contained in a maize-derived expansin (SEQ ID NO: 1) and anti-egg-white lysozyme camelid single-domain VHH antibodies was chemically synthesized in the manner described above. The nucleotide sequence and the amino acid sequence each encoding the anti-egg-white lysozyme camelid single-domain VHH antibodies used in this Example are shown in SEQ ID NOS: 9 and 10, respectively. In addition, Genst, ED. et, (2005), PNAS, 103(12), pp. 4586-4591 can be referred to for the anti-egg-white lysozyme camelid single-domain VHH antibodies used in this Example.

In this Example, the vector used in Example 1, pyr4-cbh1-no.sig-pUC(IF), was also used. The chemically synthesized polynucleotide encoding a fusion protein was inserted at the KpnI and Spel sites of the above vector. Then, the vector was formed into a linear vector by restriction enzyme treatment using Sse8387I as in the case of Example 1, followed by transformation of Trichoderma reesei. Note that the Trichoderma reesei TU6 strain was used as a host (Trichoderma reesei) in this Example.

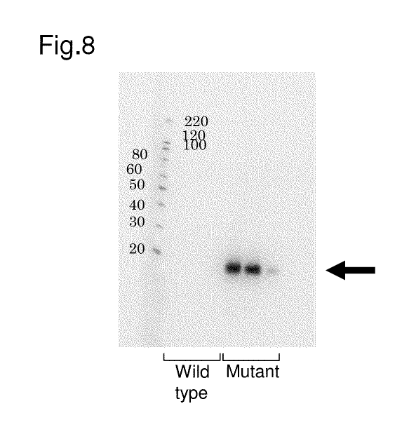

In addition, FIG. 8 shows results of Western blotting analysis using SDS-PAGE of the culture supernatant obtained by culturing transformed Trichoderma reesei as in the cases of Examples 1 and 2. For Western blotting analysis shown in FIG. 8, an anti-His6 Rabbit antibody was used as a primary antibody, and an anti-Rabbit Goat antibody was used as a secondary antibody. Further, the lane for the wild type corresponds to the culture supernatant of a nontransformed Trichoderma reesei, and the lane for a mutant corresponds to the culture supernatant of transformed Trichoderma reesei in FIG. 8.

As shown in FIG. 8, it was found that the culture supernatant of transformed Trichoderma reesei produced in this Example contained anti-egg-white lysozyme camelid single-domain VHH antibodies (1ZV5). The results revealed that the signal peptide contained in a maize-derived expansin can sufficiently function as a secretory signal peptide in a microorganism belonging to the genus Trichoderma such as Trichoderma reesei used as a host, and thus it can cause secretory production of even a mammal-derived protein.

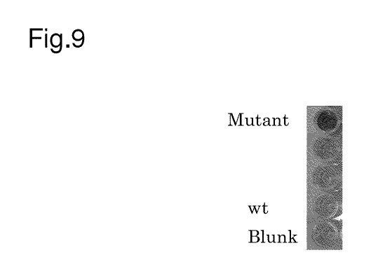

In addition, ELISA was performed using the culture supernatant of transformed Trichoderma reesei in this Example. FIG. 9 shows the results of ELISA analysis. Here, wells were coated with hen egg-white lysozyme, an anti-His6 Rabbit antibody was used as a primary antibody, and an anti-Rabbit Goat antibody was used as a secondary antibody for ELISA analysis shown in FIG. 9. Further, in FIG. 9, “Wt” denotes a well showing the result obtained using the culture supernatant of nontransformed Trichoderma reesei, and “Mutant” denotes a well showing the result obtained using the culture supernatant of transformed Trichoderma reesei.

As shown in FIG. 9, the anti-egg-white lysozyme camelid single-domain VHH antibodies (1ZV5) secreted in the culture supernatant of the transformed Trichoderma reesei produced in this Example was found to be capable of functioning to recognize and bind to an epitope of an antigen. This suggests that the conformation of the antibody can be perfectly maintained. The results revealed that the use of that microorganism belonging to the genus Trichoderma such as Trichoderma reesei as a host allows secretory production of a mammal-derived protein capable of functioning in the host.

1. A mutant microorganism belonging to the genus Trichoderma, which is obtained by transforming a microorganism belonging to the genus Trichoderma with a polynucleotide encoding a maize-derived expansin signal peptide and a protein of interest.

2. The mutant microorganism belonging to the genus Trichoderma according to claim 1, wherein the microorganism belonging to the genus Trichoderma is Trichoderma reesei.

3. The mutant microorganism belonging to the genus Trichoderma according to claim 1, wherein the signal peptide comprises the amino acid sequence shown in SEQ ID NO: 1.

4. The mutant microorganism belonging to the genus Trichoderma according to claim 1, wherein the protein of interest is a maize-derived expansin.

5. The mutant microorganism belonging to the genus Trichoderma according to claim 1, wherein the protein of interest is cellobiohydrolase, which cleaves a cellulose chain from the nonreducing end in cellobiose-unit-sized pieces.

6. The mutant microorganism belonging to the genus Trichoderma according to claim 1, wherein the protein of interest is an antibody.

7. A method for producing a protein of interest comprising culturing the mutant microorganism belonging to the genus Trichoderma according to any one of claims 1 to claim 1 in a medium so as to cause secretory production of the protein of interest.


SEQUENCE LISTING (4 sequences)

SEQ ID NO: 1 - protein, 24 aa, Zea mays
MGSLVNNIMVVGAVLAALVAGGSC

SEQ ID NO: 2 - DNA, 72 bp, Artificial Sequence
  Synthetic DNA
atgggaagccttgtcaacaacatcatggtcgtgggcgccgttctcgcggccctggtcgctggcggttcctgc

SEQ ID NO: 3 - protein, 245 aa, Zea mays
GPPKVPPGPNITTNYNGKWLTARATWYGQPNGAGAPDNGGACGIKNVNLPPYSGMTACGNVPIFKDGKGCGSCYEVRCKEKPECSGNPVTVYITDMNYEPIAPYHFDLSGKAFGSLAKPGLNDKIRHCGIMDVEFRRVRCKYPAGQKIVFHIEKGCNPNYLAVLVKYVADDGDIVLMEIQDKLSAEWKPMKLSWGAIWRMDTAKALKGPFSIRLTSESGKKVIAKDVIPANWRPDAVYTSNVQFY

SEQ ID NO: 4 - protein, 17 aa, Trichoderma reesei
MYRKLAVISAFLATARA